(19) United States
(12) Patent Application Publication
(10) Pub. No.: US 2025/0283877 A1
(43) Pub. Date: Sep. 11, 2025

(54) AUXILIARY ELECTRODE TO INCREASE THE SIGNAL FROM SILICON PHOTOELECTRIC SENSORS

(71) Applicant: Advanced Silicon Group. Inc., Lincoln, MA (US)

(72) Inventors: Nguyen Tan Khanh Le, Westford, MA (US); Nicholas Paul Turner Bateman, Reading, MA (US); Celeste F. Bedard, North Chelmsford, MA (US); Balaadithya Uppalapati, Lowell, MA (US); Marcie R. Black, Lincoln, MA (US)

(21) Appl. No.: 19/060,176

(22) Filed: Feb. 21, 2025

Related U.S. Application Data
(60) Provisional application No. 63/561,569, filed on Mar. 5, 2024.

(51) Int. Cl. G01N 33/551; G01N 27/327; G01N 33/53; G01N 33/68

(52) U.S. Cl. CPC G01N 33/551; G01N 27/327; G01N 33/5302; G01N 33/6803

(57) ABSTRACT

Provided is a biosensor with an auxiliary electrode that can increase the photocurrent signal of the biosensor due to the presence of analytes of interest.

### CROSS-REFERENCE TO RELATED APPLICATIONS

This application claims priority under 35 U.S.C. § 119(e) to U.S. Provisional Patent Application Ser. No. 63/561,569, titled “AN AUXILIARY ELECTRODE TO INCREASE THE SIGNAL FROM SILICON PHOTOELECTRIC SENSORS,” filed Mar. 5, 2024, the entire contents of which is incorporated herein by reference for all purposes.

### BACKGROUND

There exists a great desire for low-cost rapid biosensors. As we recover from the COVID pandemic, everyone has become aware of the need for reliable, easy to use, and low-cost protein sensing. Protein sensors are also used for many other medical diagnostics.

Protein sensing in the medical field is limited by the ease of use, the availability of low-cost tests, and the reliability of these tests.

Biosensors can measure nucleic acids, proteins, and other antigens for different purposes. The biosensing market is vast and includes biomanufacturing, agriculture, tissue and organ growth, and elite sports.

While there are many types of biosensors in the market and even more being developed by research groups, none of them meet all of the market needs simultaneously. So many markets remain without a viable sensing method.

A promising sensing modality is using photocurrent generated by a photovoltaic device and the change in photocurrent generated by the photovoltaic device when solutions with an antigen of interest are present vs. when they are not present.

An unpassivated or weakly passivated surface of a photovoltaic device, also referred to as a solar cell herein, serves as a very effective recombination site for photocarriers. Since the surface of the solar cell represents a disruption of the crystal lattice, the surfaces of the solar cell are sites of exceptionally high recombination. The high recombination rate in the vicinity of a surface depletes this region of minority carriers.

One measurement of the recombination rate at a surface is the surface recombination velocity. In a surface with no recombination, the movement of carriers toward the surface is zero; hence, the surface recombination velocity is zero. In a surface with infinitely fast recombination, the movement of carriers toward this surface is limited by the maximum velocity the carriers can attain.

There are different ways to increase the passivation of a silicon surface of a solar cell. One way is by putting charged particles near the surface. These charges repel like charges and can alter the passivation of the silicon surface.

Proteins and biomarkers often have a negative charge, and thus, their presence at an unpassivated or weakly passivated silicon surface can alter the surface passivation. The quality of surface passivation affects performance of the solar cell. Furthermore, the magnitude of the change in the passivation of the silicon surface can be used to deduce the number of analytes present.

In this technology, a p-n junction in a silicon wafer is functionalized with a linker, where the linker can be, for example, an antibody, aptamer, or a single strand nucleic acid. When a solution with a protein (antigen) of interest is present, that protein will bind to the linker. In some instances, the protein can bring with it an electrical charge.

The electrical charge that is now bound to the surface of the silicon through the linker will create an electric field inside the silicon and repel like charges. So, for example, if a positively charged particle is attached to the surface of the silicon, it will repel holes inside the silicon from the surface. If the surface of the silicon is n-type doped, such as in an n+/p diode, the surface will become depleted of minority carriers (holes).

When light is incident onto the silicon surface, it creates electron-hole pairs. If those electron-hole pairs separate and are collected out of the contacts of the device, they create photocurrent. However, if the electrons and holes find each other and recombine, they will not contribute to photocurrent. The surface is very effective at letting the electrons and holes recombine. In addition, silicon nanowires on the surface of the silicon have a very large surface area to volume ratio and are, therefore, very sensitive to their environment.

When the silicon surface is depleted of minority carriers in the presence of a protein, photocarriers are less likely to find each other and recombine and thus will make more photocurrent when the protein is present. In other words, the presence of the protein alters the surface recombination velocity, and thus, the photocurrent can be used to measure the presence and concentration of proteins in a solution in contact with a silicon surface of a biosensor as disclosed herein.

Thus, by shining a light that is absorbed on the silicon surface (ex. nanowire array or texture) of a solar cell and sensing the change in the electrical signal with illumination (ex. photocurrent), one can determine the concentration of an analyte attached to the silicon surface. The solar cell may thus be used as a biosensor. In some embodiments, if no analytes are attached, the silicon surface of a solar cell utilized as a biosensor will be weakly passivated, and there will be a minimal optical response. Otherwise, the silicon surface of the solar cell/biosensor will be well passivated, and the photo-created carriers may be collected from the solar cell/biosensor, leading to a strong optical response.

Embodiments of such a biosensor design were disclosed in U.S. patent application Ser. No. 18/223,765 and further refined in U.S. Pat. No. 11,585,807.

A nanowire can have a high surface area to volume ratio and, therefore, has characteristics conducive to making a very sensitive detector. Each individual nanowire of a nanowire array may be defined by a longitudinal dimension and a vertical dimension. In certain examples, the longitudinal dimension of each nanowire is at least two times longer than the vertical dimension. Accordingly, vertical arrangement of the nanowires in a nanowire array allows the nanowire array to have a significantly increased density of individual nanowires (e.g., at least 1,000 nanowires per cm²) compared to horizontal arrangements of nanowires. Such an arrangement can significantly improve the sensitivity of the biosensor.

Alternating photocurrent has been used to measure for biomarkers bound to silicon. For example, Hafeman 1988 describes using a silicon substrate covered with 1 um of insulating silicon nitride. The silicon is then attached with discrete chemistries and modulated light is shone on the surface of the silicon. By looking at the alternating current signal from the change in capacitance of the silicon-insulator device, the authors can determine if the chemistry is active (for example, the pH of the solution).

### SUMMARY

In an embodiment of the disclosure, the photocurrent signal from a silicon p-n junction biosensor is enhanced by applying an external electric field to the solution of interest.

In accordance with one aspect, there is provided a sensor with a semiconductor device, the semiconductor device having an internal electric field, a linker attached to a surface of the semiconductor device, a well to hold a solution of interest, and an auxiliary electrode touching the solution and not directly touching the semiconductor.

In some embodiments, the semiconductor device is formed of silicon.

In some embodiments, the internal electric field is formed by a p-n junction.

In some embodiments, the auxiliary electrode is a conducting electrode that sits on the silicon to a side of the p-n junction.

In some embodiments, the auxiliary electrode is a transparent conducting electrode that sits on top of the well.

In some embodiments, the linker is one of an antibody, aptamer, or nucleic acid.

In some embodiments, the linker is chosen to measure a protein or proteins of interest.

In some embodiments, the proteins of interest are host cell proteins.

In accordance with another aspect, there is provided a method of determining the presence or amount of an analyte within a solution of interest with a semiconductor device. The method comprises measuring a first photocurrent generated by the semiconductor device when exposed to a predetermined frequency and intensity of optical illumination in absence of the analyte, exposing a surface of the semiconductor device to the solution of interest, applying a voltage to an auxiliary electrode that is in contact with the solution of interest and not in direct electrical contact with the surface semiconductor device, the auxiliary electrode applying an electric field evenly to the surface of the semiconductor device, measuring a second photocurrent generated by the semiconductor device with the surface of the semiconductor device exposed to the solution of interest, the voltage applied by the auxiliary electrode, and the semiconductor device is exposed to the predetermined frequency and intensity of optical illumination, and determining the presence or amount of the analyte within the solution of interest based on a comparison between the first photocurrent and the second photocurrent.

### BRIEF DESCRIPTION OF THE FIGURES

FIG. 1 is a picture of an example of a biosensor as disclosed herein including a polydimethylsiloxane (PDMS) well.

FIG. 2 is a schematic plane view of an example of a biosensor as disclosed herein.

FIG. 3 illustrates a cross section of a biosensor as disclosed herein during a test with the electrode on top. The cross section is shown in a region of the biosensor without the front contact.

FIG. 4 is a chart of the photocurrent of an example of a biosensor as disclosed herein in pH5 and pH9 buffers when the voltage between the auxiliary electrode and the top electrode is 0V and 1.2V.

FIG. 5A is a chart of the normalized external quantum efficiency curves for an example of a biosensor as disclosed herein in pH5 and pH9 buffers when the voltage between the auxiliary electrode and the top electrode is 0V.

FIG. 5B is a chart of the normalized external quantum efficiency curves for an example of a biosensor as disclosed herein in pH5 and pH9 buffers when the voltage between the auxiliary electrode and the top electrode is 1.2V.

FIG. 6 is an example of a design of a biosensor as disclosed herein including an auxiliary electrode disposed on the silicon substrate of the biosensor.

### DETAILED DESCRIPTION

Before describing aspects and embodiments disclosed herein in detail, it must be understood that the aspects and embodiments disclosed herein are not limited to specific solvents, materials, or device structures, as such may vary. It is also to be understood that the terminology used herein is to describe particular embodiments only, and is not intended to be limiting.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also disclosed, as well as the range of values greater than or equal to 1 μm and the range of values less than or equal to 8 μm.

Examples of the systems and methods discussed herein are not limited in application to the details of construction and the arrangement of components outlined in the following description or illustrated in the accompanying drawings. The systems and methods are capable of implementation in other embodiments and of being practiced or carried out in various ways. Examples of specific implementations are provided herein for illustrative purposes only and are not intended to be limiting. In particular, acts, components, elements, and features discussed in connection with any one or more examples are not intended to be excluded from a similar role in any other examples.

Also, the phraseology and terminology used herein is for the purpose of description and should not be regarded as limiting. Any references to examples, embodiments, components, elements, or acts of the systems and methods herein referred to in the singular may also embrace embodiments, including a plurality, and any references in plural to any embodiment, component, element, or act herein may also embrace embodiments including only a singularity. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof, as well as additional items. References to “or” may be construed as inclusive so that any terms described using “or” may indicate any of a single, more than one, and all of the described terms. In addition, in the event of inconsistent usages of terms between this document and documents incorporated herein by reference, the term usage in the incorporated features is supplementary to that of this document; for irreconcilable differences, the term usage in this document controls.

Often, a measurement for an analyte, biomarker, or antigen will be done in solution. The solution could be a buffer solution or a solution with a set pH level, for example.

A biosensor can be used in a way that the photocurrent generated by a photovoltaic cell of the biosensor is measured. The photocurrent can be measured as a function of wavelength or with and without the solution of interest present.

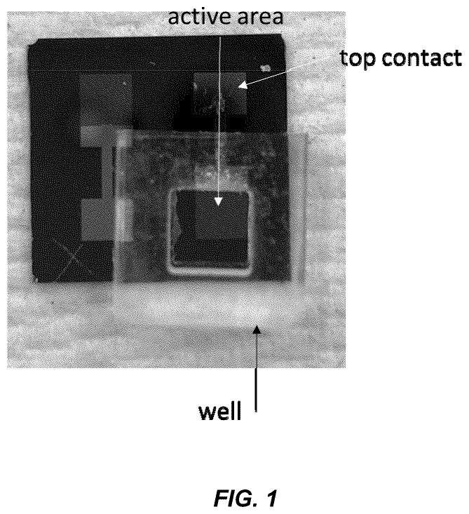

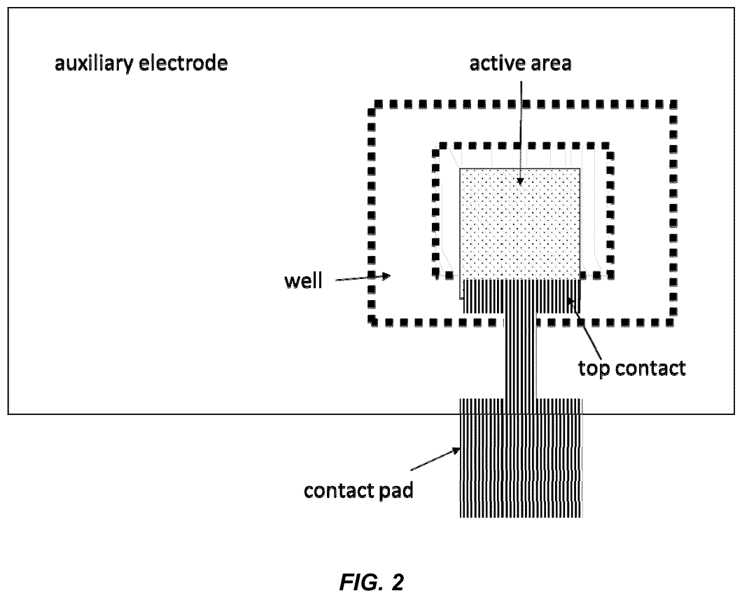

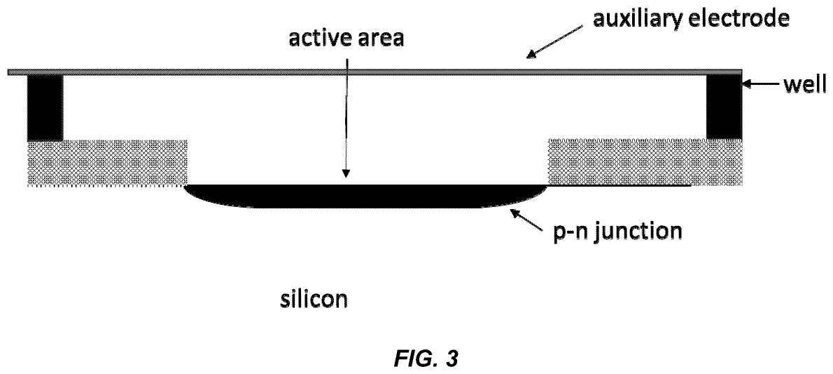

To hold the solution of interest over the sensing area, a well can be used. One way to make such a well is by using a mold and PDMS, which are well-known in the industry. The well can be attached to a silicon surface of the photovoltaic cell of the biosensor by putting the well on the silicon surface and placing the biosensor on a hot plate at 100° C. for 60 minutes. An examples of a biosensor with a PDMS well attached to the silicon surface of the photovoltaic cell of the biosensor is illustrated in FIG. 1. FIG. 2 shows a schematic of a biosensor with a well attached to the biosensor surface and with an auxiliary electrode over the biosensor and well. FIG. 3 shows a cross-sectional schematic of the biosensor of FIG. 2.

Solution can then be added to the well, and measurements taken with and without light incident through the solution onto the top of the sensor.

One challenge to doing a measurement that depends on the electric charge and that is done in a solution is that solutions (ex. buffer solutions) can have ions present. These ions can move around in the solution, and screen charges present on the silicon surface of the photovoltaic cell of the biosensor. This will decrease the electric field at the silicon surface and, hence, the signal generated by the biosensor.

The distance of screening is referred to the Debye length. The Debye length decreases with more ions in the solution.

Applying a voltage between an electrode in the solution and the top electrode of the biosensor (the electrode attached to the silicon surface of the photovoltaic cell of the biosensor) can create an electric field in the solution. An electric field near the surface of the biosensor where charged antigens are bonded will move any mobile charges (ex. ions) in the solution. If the electric field is in the right direction, ions that are counteracting the field of the bounded and charged antigens can be pulled away, and thus, the screening of the charge from the bound antigens may be decreased.

The decrease in the screening of the charged antigens bound to the silicon surface will alter the photocurrent generated by the biosensor. It can increase the photocurrent signal and the change in the photocurrent as a result of the presence of the charged antigens. This increase in signal is desired for more sensitive and reproducible measurements.

A bias to the solution can be applied with a probe similar to the controlling electrode in reference to “Light-addressable Potentiometric sensor for Biochemical Systems,” where the sensor is a silicon/silicon nitride/solution capacitor (no junction in the silicon) with LED excitation and where an AC photocurrent is measured. However, this probe is localized and thus produces a nonuniform electric field on the surface of the sensor. Often, an electrode that applies a voltage more uniform to the solution is desired. For example, the probe could cause shadowing and make reproducible alignment difficult, while alignment might be easier with an electrode coving the whole well uniformly.

Also, the contact area of the localized probe depends on how deep the probe is in the solution, while a more uniform top electrode has a contact area equal to the cross-sectional area of the well. The well has a more controlled and repeatable contact area and the distance to the surface of the biosensor is controlled by the well height.

Furthermore, when the distance between the electrode and the silicon surface of the photovoltaic cell of the biosensor is smaller than twice the Debye length in the solution, there is a significant electric field in the solution and a voltage drop throughout the bulk of the solution. Accordingly, the voltage at the surface of the silicon depends on the distance between the electrode and the surface of the silicon. A probe will therefore have an uneven electric field on the sensor surface while a planar electrode will have a more even surface.

The Debye length is the distance that the electric field from an electric charge is seen in the solution. Beyond this distance, the electric field is screened by the ions in the solution. Thus, in smaller volumes (for example in microfluidics or microwells) the electric field is more likely to penetrate through more of the solution. Furthermore, solutions with less ions have larger Debye lengths. With smaller volumes and/or less ions, an electrode can be placed over the well of the device holding the solution and alter the electric field at the surface of the biosensor.

While light excitation can be done from the rear of the biosensor device, there are advantages to exciting through the top of the biosensor device. In particular, light with a high optical absorption can be incident through the top of the biosensor. Examples of biosensors disclosed herein may be more sensitive when utilized with light absorbed at surface of the silicon than light that is absorbed deeper into the silicon. Blue light, for example, has a higher absorption coefficient than red light.

While light excitation from the top of the biosensor device is desirable in some implementations, and having an auxiliary electrode that more uniformly applies bias than a probe is preferred, an opaque electrode will not let light be incident onto the front surface of the biosensor device. Hence, an opaque electrode on top of the biosensor and exciting from the top will not result in photocurrent generated in the biosensor.

A transparent or translucent electrode can instead be used to pass the light through the electrode. Transparent electrodes can be made from transparent conducting oxides or thin metal deposited onto a glass slide. The work function and the charge transfer to the solution should be considered in the selection of the electrodes.

Thin transparent metal can be made from gold, platinum, aluminum, or other metals. In addition, a two-stack metal may be desired, such as titanium for an adhesion layer and another metal for the electrical contact or two metals in the list above.

FIG. 3 illustrates an example of placement of the auxiliary electrode.

In one embodiment 400 nm of titanium and then 400 nm of platinum are evaporated onto a glass slide to form the auxiliary electrode. In another, titanium and gold are used.

The height of the well can be selected both for the amount of solution desired to be exposed to the biosensor and to have a fixed distance of the auxiliary electrode to the surface of the biosensor. For example, the well height can be 1 mm, 4 mm, or 10 mm, or another height selected as desired for a particular implementation.

As an alternative to placing a transparent conductor on top of the well, an auxiliary electrode can be placed on the surface of the silicon chip that the biosensor is made from. In this sensor design, there are three electrical contacts on the silicon chip—one for the emitter (n+ or p+ region), one for the lighter doped side of the p-n junction (base), and a third for the auxiliary electrode.

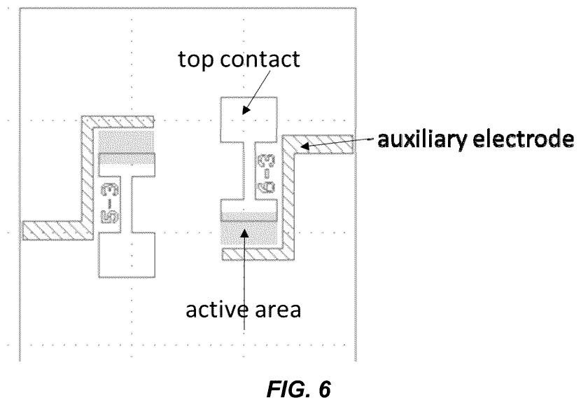

FIG. 6 shows an example of a sensor design with the auxiliary electrode to the side of other portions of the biosensor.

The auxiliary electrode does not have to be disposed on the same semiconductor chip from which remaining portions of the biosensor are formed. For example, portions of the biosensor can be removed from the larger wafer in which it was manufactured and placed onto a circuit board. The auxiliary electrode can be attached onto the circuit board to the side of the active region of the biosensor.

The auxiliary electrode is not electrically connected to the other two contacts and is electrically connected to the solution in the well.

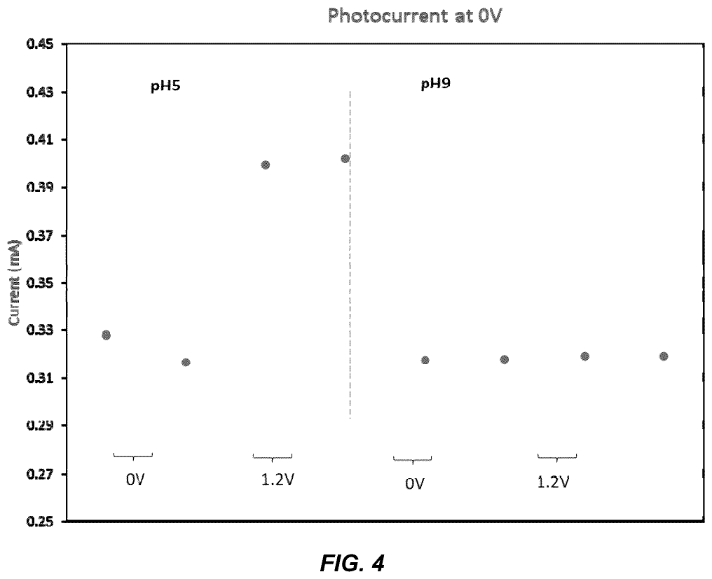

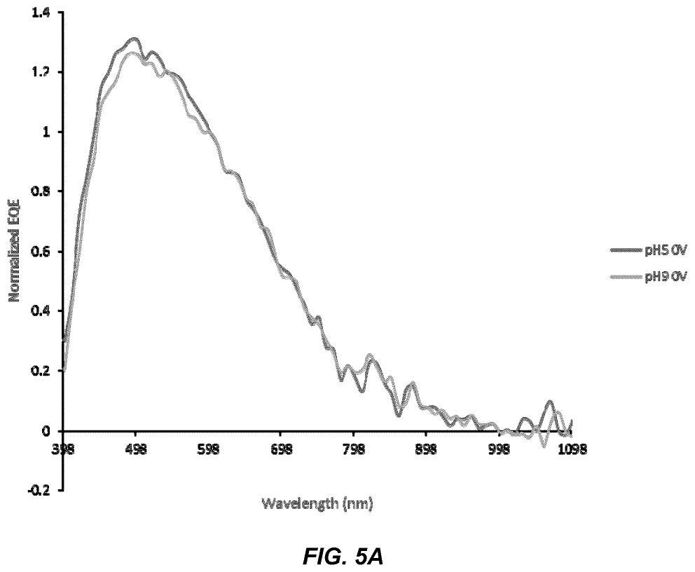

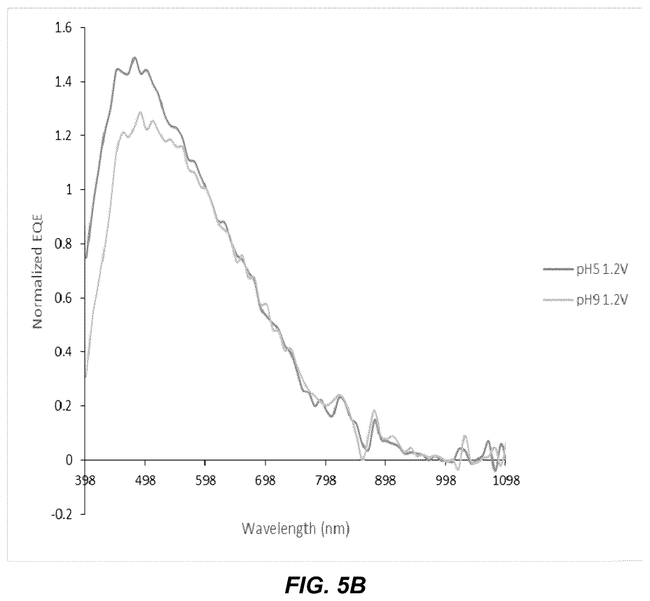

FIGS. 4, 5A, and 5B show the experimental results of an example of a biosensor as disclosed herein with an applied auxiliary electrode. In this embodiment, the sensor is functionalized with APTES. When in a buffer with pH 5, the APTES has a positive charge. The sensor has a p-n junction with an n+ phosphorous emitter and a base silicon with lightly p-type doping. Thus, the positively charged APTES repels the minority carriers in the front side emitter.

When the sensor is exposed to the buffer of pH 9, the APTES is not charged.

FIGS. 5A and 5B show the normalized external quantum efficiency (EQE) of the biosensor when exposed to pH9 and pH5 buffers with 0V and 1.2V applied to the external buffer relative to the front side emitter. The EQE shows an increased signal in blue light when a voltage is applied to the auxiliary electrode and the biosensor device is exposed to pH5. In pH9, the APTES does not have a significant charge, while in pH5, it will have a positive charge. The signal in the blue light is only enhanced when a voltage is applied as the electric field due to the applied voltage to the auxiliary electrode decreases screening and thus increases the signal. The signal is observed in blue light and not in infrared light because blue light absorbs closer to the surface in silicon of the biosensor, while infrared light absorbs deep into the silicon and thus is less sensitive to the surface. Light absorbed below the junction depth is less sensitive to the surface.

The surface of the biosensor may include silicon nanowire arrays, which will increase the surface area to volume ratio of the surface of the biosensor, making the biosensor device more sensitive to the antigens in the solution.

The p-n junction can act as a photovoltaic cell and operated in the region of power generation. It can also be operated in reverse bias, not in the region of power production. The power generated by the biosensor can be used, for example, to communicate the measurement or to charge a battery.

More generally, the biosensor can be made with a built-in field for methods other than a p-n junction. Also the field can be not just at the surface of the device but also inside the silicon, as is the case for a p-n junction in silicon.

The emitter depth can be 100 nm, 300 nm, or 1 um, or at other depths selected as desired for a particular implementation. Alternatively, the emitter can be on the back-side of the device.

The photocurrent can be measured as a function of the applied auxiliary electrode voltage. The shape of how the photocurrent changes as a function of the applied voltage to the auxiliary electrode, such as how fast the photocurrent changes and at what applied voltage it changes, can provide information about the bound antigens.

The applied voltage can also be an alternating current with a set frequency. The frequency of that applied voltage, as well as the centered voltage that the AC voltage is on top of can be varied. Certain frequencies (ex. 10 kHz to 100 kHz) can be particularly useful as the mobile ions are limited to how fast they can move in the solution.

Instead of using an auxiliary electrode to apply a voltage and create a charge that pulls away mobile charges from the surface of the silicon, another embodiment allows for the well itself to hold an electric charge. A charge on the well will also repel and attract mobile charges in the solution and, therefore, can be used to increase the signal by decreasing screening from mobile charges.

Some embodiments include large arrays of nanowires, and some aspects include improvements to the biosensor disclosed in U.S. Pat. No. 11,585,807.

An array of nanowires maybe formed on the surface of a biosensor as disclosed herein. The biosensor of various embodiments maybe constructed by fabricating at least one nanowire array, forming a photovoltaic cell by doping the top surface of a substrate, electrically contacting the substrate to the nanowire array, and functionalizing (e.g., chemically coating) the nanowires. The nanowires can be incorporated into a biosensor, which may be exposed to a sample to determine the presence, absence, or concentration of an analyte within the sample.

As discussed herein, functionalization may refer to coating a silicon surface with a desired chemical that is sensitive to an analyte (e.g., a biomarker binding agent) or protein. In many instances, this functionalization results from attaching an antibody or aptamer to the silicon surface. The antibody or aptamer selectively binds to other organic material, such as a protein. When functionalized, the silicon surface of a biosensor may be exposed to the protein or analyte for which it is functionalized; the electrical properties may change. Suppose subsensors, e.g., different regions of a biosensor as disclosed herein, are functionalized for different biological materials. In that case, the electrical properties of each subsensor will change differently depending on the concentration in the measured solution of the particular material it is functionalized for.

Analytes can be biomarkers, proteins, DNA, or any material that one can make to selectively attach to a functionalized silicon surface.

The biosensor device design can also be used with planar silicon or microtextured silicon, but the photoelectric response of nanowires/nanotexture can be especially dependent on the quality of surface passivation since they have a high surface area. For example, the surface area of a nanowire array may be over a hundred or a thousand times greater than that of a flat surface or a single nanowire device. As a result of the high surface to volume ratio, nanotextured or nanowire surfaces can be used in a biosensor and result in detection of analytes with a lower level of concentration. The lower threshold of detection can be as low as 10 ng/ml, 1 pg/ml, or even in some implementations, 1 fg/ml. According to various examples, the biosensor may utilize the sensitivity of electrical properties of the nanowire array, in particular, the quality of the front surface passivation. For example, the nanowire biosensor/solar cell can be measured in a wavelength range that is sensitive to the front surface (ex. light with wavelengths between 350 and 700 nm). Suppose the back surface (non-illuminated side) is being used to attach the analytes. In that instance, one may use wavelengths that penetrate deeper into the silicon, such as those wavelengths between 700 nm and 1100 nm.

The biosensor can be subdivided into subsensors, with each subsensor having, for example, a thousand to millions of nanowires. Each subsensor may be functionalized to detect a given analyte (e.g., a cancer biomarker, host cell protein, or mutated DNA).

The incident light can be scanned over a desired section of the nanowire array or “flashed” to illuminate the entire sample. Once illuminated, the current-voltage of each subarray is taken individually, and a measurement for each subarray group is provided. The light can be incident onto the sample from either the front side or from both sides, and different wavelengths can be used to help better understand the results, where the front refers to the side upon which the light is incident.

The silicon biosensor can be formed from either an n-type wafer doped with a p+ region or a p-type wafer doped with an n+ region. The emitter (heavily doped region) can be on either the front or back of the biosensor device. For example, the emitter can also be local as in either a selective emitter (locally doped under contacts) or interdigitated back contact design (where the two contacts are on the same side of the device). In some implementations, one might also choose to use a metal-silicon junction, in which case a heavily doped region may not be needed. A tunnel junction may also be desired where the junction forms from tunneling through a dielectric such as silicon dioxide. The tunneling can happen between a metal and silicon through silica.

### Exemplary Process

Silicon biosensors are fabricated, for example, using the process described in U.S. Pat. No. 10,079,322 and U.S. patent application Ser. No. 18/223,765.

After the sensors are fabricated, they are functionalized. This can be done by attaching an antibody to the silicon surface. For example, we first prepare a 3-(aminopropyl) triethoxysilane (APTES)/ethanol (EtOH) solution. APTES: dilute 50 ul APTES in 2.45 ml EtOH for a total of 2.5 ml per sensor. We then place the sensor into the APTES/EtOH solution for 4 hours on a rocker. Then, we wash the sensor in EtOH.

Next, we put on wells that have been premade with PDMS and a mold. To do this, we put the well on and place it on a hot plate for 60 minutes.

Next, we mount the sensor to our testing setup that electrically contacts the front and back electrodes. We then put buffer, for example, pH5, into the well and place the auxiliary electrode on top of the solution. We need to be careful that there are no bubbles in the well and that the buffer does not overflow the well and short out the contacts.

We then align the testing setup for maximum signal. We can measure the current-voltage response of the sensor both with and without light. We can also measure the quantum efficiency signal.

When the test is done, we can rinse out the buffer solution and put a different buffer solution or solution of interest inside the well.

As discussed herein, in various aspects and embodiments the biosensor may be configured to measure indications of cancer, and other illnesses. Such aspects and embodiments offer the benefits of earlier cancer detection, and less medical waste (e.g., smaller blood samples). Applications also include measuring host cell proteins or other proteins in biomanufacturing. The sensors can also be used to measure charge variation in a biopharma solution. The sensors could also be used to monitor a person's health outside a doctor's office. For example, one can measure biomarkers, proteins, or DNA in urine, spit, bowel movements, or from a pin prick and drop of blood. These biomarkers can tell if a person is pregnant, has the flu or COVID, or is suffering from food poisoning.

Sensors can be used to measure biomarkers on a regular basis and recorded as a function of time to allow the person to track changes in biomarkers. The measurement tool can be made to communicate with a computer that keeps track of measurements and the time of measurements. This connection can be wireless or wired. The measurement tool can be placed on the body or in locations such as the toilet for easy access to bodily fluids.

Tests can be used to indicate diseases, measure protein in the urine for diabetes for example, indicate times of fertility, or optimize for high performance sports training.

The term “nanostructure” as used herein, refers to a structure typically characterized by at least one physical dimension less than about 300 nm. In this nanostructure a property of interest is different than that of bulk, or not nanostructured materials.

The term “nanowire” describes a material with a shape that typically has one principle axis that is longer than another dimension which is nanostructured. Thus, they have an aspect ratio greater than one and often greater than 2 or 5. In certain embodiments, nanowires herein have a substantially uniform diameter. In some embodiments, the diameter shows a variance along the axis of the wire. In some embodiments, the wires have a roughly circular cross section, but in other embodiments, the cross section is oval or other non-circular shapes.

It will be appreciated that the term “nanostructure” can include structures such as nanopyramids, nanowires, nanotubes, nanopores, and other nanosized features.

The term “functionalized” refers to the process of attaching one or more functional moiety (antibody, antigen, ligand, etc.) such as a chemically reactive group to the surface. The nano-surface us functionalized to for example confer specificity for a desired analyte in a reaction such as in an assay. Those skilled in the art will be aware of many different functionalization methods which can be used.

Having thus described several aspects of at least one embodiment, it is to be appreciated that various alterations, modifications, and improvements will readily occur to those skilled in the art. Such alterations, modifications, and improvements are intended to be part of this disclosure and are intended to be within the spirit and scope of the aspects and embodiments disclosed herein. Accordingly, the foregoing description and drawings are by way of example only.

The following references may be relevant to this application and the subject matter of same incorporated herein by reference: (1) Sami Franssila, Introduction to Microfabrication (2d ed. John Wiley & Sons 2010). (2) U.S. Published Patent Application No. 2009/256134. (3) U.S. Pat. No. 8,852,981. (4) H. Galinski et al., “Agglomeration of Pt thin films on dielectric substrates,” Phys. Rev. B, 82, 235415 (2010). (5) Feng-Ming Liu & Mino Green, “Efficient SERS substrates made by electroless silver deposition into patterned silicon structures,” J. Mater. Chem., 14, 1526-1532 (2004). (6) D. W. Pashley et al., “The growth and structure of gold and silver deposits formed by evaporation inside an electron microscope,” Phil. Mag., 10:103, 127-158 (1964). (7) Muller, Richard S., Theodore I. Kamins, Mansun Chan, and Ping K. Ko. “Device electronics for integrated circuits.” (1986): 54. (8) Weste, Neil HE, and Kamran Eshraghian. “Principles of VLSI Design.” A Systems Perspective 2 (1985). (9) U.S. Pat. No. 8,450,599. (10) U.S. Pat. No. 8,143,143. (11) U.S. Pat. No. 10,079,322, (12) patent application Ser. No. 18/223,765, (13) U.S. Pat. No. 11,585,807, (14) Hafeman, Dean G., J. Wallace Parce, and Harden M. McConnell. “Light-addressable potentiometric sensor for biochemical systems.” Science 240, no. 4856 (1988): 1182-1185. (15) Dresselhaus, Mildred. Yu-Ming Lin, Oded Rabin, Marcie Black, Jing Kong, and Gene Dresselhaus. “Nanowires.” Springer Handbook of Nanotechnology (2007): 113.

All patents, patent applications, and publications mentioned in this application are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

What is claimed is:

1. A sensor with a semiconductor device, the semiconductor device having an internal electric field, a linker attached to a surface of the semiconductor device, a well to hold a solution of interest, and an auxiliary electrode touching the solution and not directly touching the semiconductor.

2. The sensor of claim 1, where the semiconductor device is formed of silicon.

3. The sensor of claim 2, where the internal electric field is formed by a p-n junction.

4. The sensor of claim 3, where the auxiliary electrode is a conducting electrode that sits on the silicon to a side of the p-n junction.

5. The sensor of claim 1, where the auxiliary electrode is a transparent conducting electrode that sits on top of the well.

6. The sensor of claim 1 where the linker is one of an antibody, aptamer, or nucleic acid.

7. The sensor of claim 6, where the linker is chosen to measure a protein or proteins of interest.

8. The sensor of claim 7, where the proteins of interest are host cell proteins.

9. A method of determining the presence or amount of an analyte within a solution of interest with a semiconductor device, the method comprising:
measuring a first photocurrent generated by the semiconductor device when exposed to a predetermined frequency and intensity of optical illumination in absence of the analyte;
exposing a surface of the semiconductor device to the solution of interest;
applying a voltage to an auxiliary electrode that is in contact with the solution of interest and not in direct electrical contact with the surface semiconductor device, the auxiliary electrode applying an electric field evenly to the surface of the semiconductor device;
measuring a second photocurrent generated by the semiconductor device with the surface of the semiconductor device exposed to the solution of interest, the voltage applied by the auxiliary electrode, and the semiconductor device is exposed to the predetermined frequency and intensity of optical illumination; and
determining the presence or amount of the analyte within the solution of interest based on a comparison between the first photocurrent and the second photocurrent.
